(19) United States
(12) Patent Application Publication
(10) Pub. No.: US 2013/0190841 A1
(43) Pub. Date: Jul. 25, 2013

(54) PRESCRIPTION DEVICE CONTROLLER

(71) Applicant: William C. McMillan, Dunwoody, GA (US)

(72) Inventor: William C. McMillan, Dunwoody, GA (US)

(21) Appl. No.: 13/671,738

(22) Filed: Nov. 8, 2012

Related U.S. Application Data
(63) Continuation of application No. 12/717,516, filed on Mar. 4, 2010, now abandoned.

(51) Int. Cl. A61N 5/06

(52) U.S. Cl. CPC A61N 5/0616
USPC 607/88

(57) ABSTRACT

A device for controlling ultraviolet phototherapy exposure to a patient is provided. The phototherapy device has an ultraviolet light source, a biometric scanner and a phototherapy computer coupled together. A prescribing physician can prescribe at least one phototherapy setting for the patient which can be transmitted to the remote phototherapy computer. In order to activate the ultraviolet light source according to the phototherapy setting, the patient can be required to input into the phototherapy computer a patient passcode, and the patient must undergo a biometric scan from the biometric scanner to verify the correct patient is receiving treatment.

### CROSS-REFERENCE TO RELATED APPLICATIONS

This application is a continuation of copending U.S. application Ser. No. 12/717,516, filed Mar. 4, 2010, which is hereby incorporated herein by reference in entirety.

### FIELD OF THE INVENTION

Provided are methods and apparatuses for controlling ultraviolet phototherapy exposure to a patient. More specifically, provided are methods and apparatuses for controlling ultraviolet phototherapy exposure to a patient remote from a physician.

### BACKGROUND

Ultraviolet light is one of the most common forms of radiation that humans are exposed to on a regular basis. It is also one of the most damaging, with its effects cumulative. However, ultraviolet light is also an effective method of therapy for several chronic skin conditions. Ultraviolet phototherapy employs relatively intense ultraviolet light exposure in a controlled manner. It is economical, easy to deliver and has readily overcome side effects. The risks from properly administered and monitored therapy are minimal. However, improper use can be dangerous, leading to injuries such as sunburn-type injuries, eye damage, skin cancer, and even fatal melanomas.

For ultraviolet light therapy to be most effective, the patient should be exposed to the ultraviolet light several times per week. Exposure times vary greatly, but typically are a few seconds to several minutes. In order for the patient to follow a treatment regimen properly in a clinical setting, the patient must travel to the clinic three to five times per week, which involves transportation and related expenses, time removed from daily obligations, as well as time spent waiting in the clinic. Often, a single five minute treatment can consume several hours of a patient's time.

Physicians have been reluctant to release phototherapy equipment to a patient for remote use for fear of misuse by the patient and/or other persons, because there has been no method or device to ensure patient compliance outside of the clinical setting. Thus, there is a need in the art for an ultraviolet phototherapy device configured to control ultraviolet phototherapy exposure to a patient remote from a physician.

### SUMMARY

In accordance with the purpose(s) of this invention, as embodied and broadly described herein, in one aspect, a device for controlling ultraviolet phototherapy exposure to a patient is provided. In another aspect, the phototherapy device comprises an ultraviolet light source and a phototherapy computer coupled to the ultraviolet light source and configured to activate the ultraviolet light source. In another aspect, the phototherapy device further comprises a biometric scanner coupled to the phototherapy computer and configured for scanning at least one biometric parameter of the patient.

In one aspect, a prescribing physician can prescribe at least one phototherapy setting for the patient which can be transmitted to the phototherapy computer. In another aspect, in order to activate the ultraviolet light source according to the at least one phototherapy setting, the patient can be required to input into the phototherapy computer a patient passcode, and the patient must undergo a biometric scan from the biometric scanner. If the patient inputs the proper patient passcode, and the results of the biometric scan are positive, the ultraviolet light source can be activated, according to another aspect.

In one aspect, the at least one phototherapy setting is transmitted to the phototherapy device electronically over a network. In another aspect, the at least one phototherapy setting is transmitted to the phototherapy device electronically via a portable storage device. After use of the device, according to another aspect, conditions of the activation of the ultraviolet light source can be logged by the phototherapy computer and can be remotely accessible to a prescribing physician electronically over a network or via a portable storage device.

The ultraviolet light source, according to one aspect, comprises at least one ultraviolet lamp, and at least one sensor configured to send a signal to the phototherapy computer representing at least one operating condition of the ultraviolet light source. In another aspect, the at least one ultraviolet lamp and the at least one sensor have an electronic identifier. In this aspect, if the proper electronic identifier is not present the ultraviolet light source can be prevented from activating. In another aspect, if the at least one sensor signals to the phototherapy computer an undesired operating condition, the ultraviolet light source can be prevented from activating.

Additional advantages will be set forth in part in the description which follows, and in part will be obvious from the description, or may be learned by practice of the aspects of the invention as described herein. The advantages can be realized and attained by means of the elements and combinations particularly pointed out in the appended claims. It is to be understood that both the foregoing general description and the following detailed description are exemplary and explanatory only and are not restrictive of the aspects of the invention, as claimed.

### BRIEF DESCRIPTION OF THE DRAWINGS

The accompanying drawings, which are incorporated in and constitute a part of this specification, illustrate aspects of the invention and, together with the description, serve to explain the principles of the invention.

FIG. 1 illustrates a simplified, non-limiting block diagram showing select components of an exemplary operating environment for performing the disclosed methods.

FIG. 2 is a perspective view of a phototherapy device of the present application, according to one aspect.

### DETAILED DESCRIPTION

The present invention may be understood more readily by reference to the following detailed description of aspects of the invention and to the Figures and their previous and following description.

The present invention may be understood more readily by reference to the following detailed description, examples, drawings, and claims, and their previous and following description. However, before the present devices, systems, and/or methods are disclosed and described, it is to be understood that this invention is not limited to the specific devices, systems, and/or methods disclosed unless otherwise specified, as such can, of course, vary. It is also to be understood that the terminology used herein is for the purpose of describing particular aspects only and is not intended to be limiting.

As used in the specification and the appended claims, the singular forms “a,” “an” and “the” include plural referents unless the context clearly dictates otherwise. Thus, for example, reference to a “lamp” can include two or more such lamps unless the context indicates otherwise.

Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

As used herein, the terms “optional” or “optionally” mean that the subsequently described event or circumstance may or may not occur, and that the description includes instances where said event or circumstance occurs and instances where it does not.

Reference will now be made in detail to the present preferred aspect(s) of the invention, examples of which are illustrated in the accompanying drawings. Wherever possible, the same reference numbers are used throughout the drawings to refer to the same or like parts.

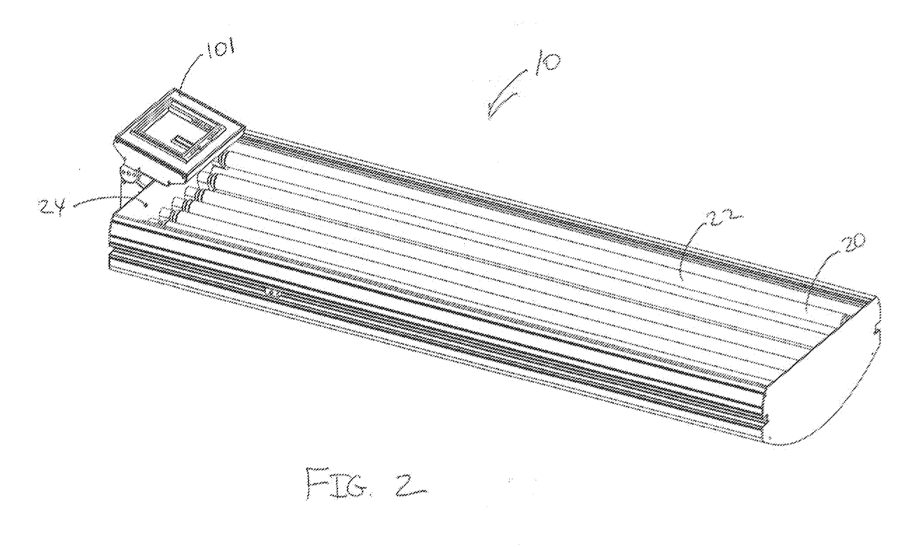

In one aspect, a phototherapy device 10 is provided for controlling ultraviolet phototherapy exposure to a patient. In another aspect, as illustrated in FIG. 2, the phototherapy device can comprise at least one of: an ultraviolet light source 20, a phototherapy computer 101, at least one sensor 24 and a biometric scanner.

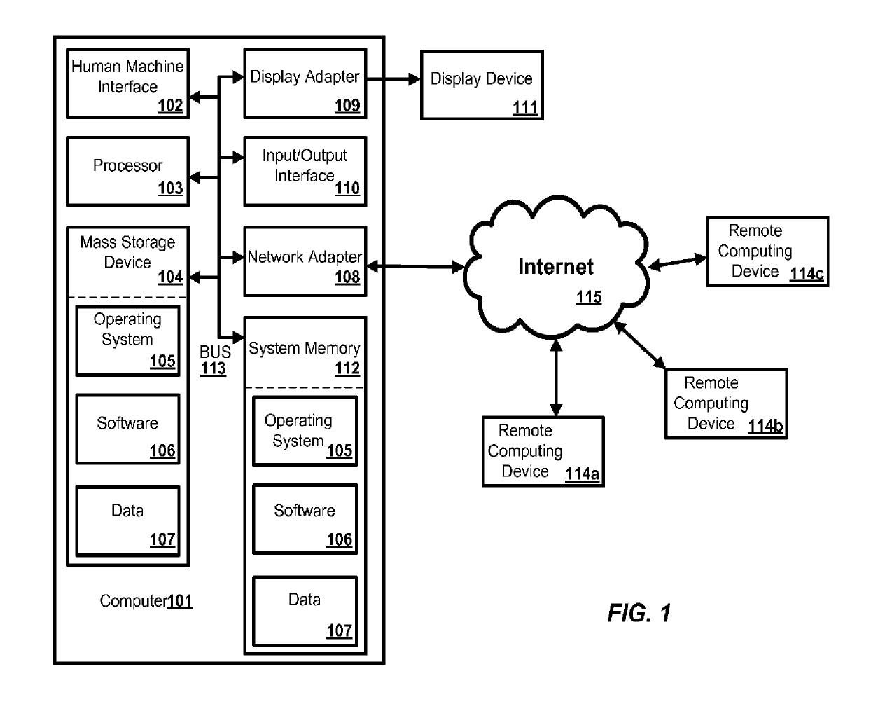

FIG. 1 is a block diagram illustrating an exemplary operating environment for performing the disclosed methods and portions thereof. This exemplary operating environment is only an example of an operating environment and is not intended to suggest any limitation as to the scope of use or functionality of operating environment architecture. Neither should the operating environment be interpreted as having any dependency or requirement relating to any one or combination of components illustrated in the exemplary operating environment.

The present methods and systems can be operational with numerous other general purpose or special purpose computing system environments or configurations. Examples of well known computing systems, environments, and/or configurations that can be suitable for use with the system and method comprise, but are not limited to, personal computers, server computers, laptop devices, hand-held electronic devices, vehicle-embedded electronic devices, and multiprocessor systems. Additional examples comprise set top boxes, programmable consumer electronics, network PCs, minicomputers, mainframe computers, distributed computing environments that comprise any of the above systems or devices, and the like.

The processing of the disclosed methods and systems can be performed by software components. The disclosed system and method can be described in the general context of computer-executable instructions, such as program modules, being executed by one or more computers or other devices. Generally, program modules comprise computer code, routines, programs, objects, components, data structures, etc. that perform particular tasks or implement particular abstract data types. In one aspect, the program modules can comprise a system control module. The disclosed method can also be practiced in grid-based and distributed computing environments where tasks are performed by remote processing devices that are linked through a communications network. In a distributed computing environment, program modules can be located in both local and remote computer storage media including memory storage devices.

Further, one skilled in the art will appreciate that the system and method disclosed herein can be implemented via a general-purpose computing device in the form of a computer 101. The components of the computer 101 can comprise, but are not limited to, one or more processors or processing units 103, a system memory 112, and a system bus 113 that couples various system components including the processor 103 to the system memory 112.

The system bus 113 represents one or more of several possible types of bus structures, including a memory bus or memory controller, a peripheral bus, an accelerated graphics port, and a processor or local bus using any of a variety of bus architectures. By way of example, such architectures can comprise an Industry Standard Architecture (ISA) bus, a Micro Channel Architecture (MCA) bus, an Enhanced ISA (EISA) bus, a Video Electronics Standards Association (VESA) local bus, an Accelerated Graphics Port (AGP) bus, and a Peripheral Component Interconnects (PCI) bus also known as a Mezzanine bus. The bus 113, and all buses specified in this description can also be implemented over a wired or wireless network connection and each of the subsystems, including the processor 103, a mass storage device 104, an operating system 105, prescription software 106, prescription and/or treatment data 107, a network adapter 108, system memory 112, an Input/Output Interface 110, a display adapter 109, a display device 111, and a human machine interface 102, can be contained within one or more remote computing devices 114a,b,c at physically separate locations, connected through buses of this form, in effect implementing a fully distributed system.

The computer 101 typically comprises a variety of computer readable media. Exemplary readable media can be any available media that is accessible by the computer 101 and comprises, for example and not meant to be limiting, both volatile and non-volatile media, removable and non-removable media. The system memory 112 comprises computer readable media in the form of volatile memory, such as random access memory (RAM), and/or non-volatile memory, such as read only memory (ROM). The system memory 112 typically contains data such as prescription and/or treatment data 107 and/or program modules such as operating system 105 and prescription module software 106 that are immediately accessible to and/or are presently operated on by the processing unit 103.

In another aspect, the computer 101 can also comprise other removable/non-removable, volatile/non-volatile computer storage media. By way of example, FIG. 1 illustrates a mass storage device 104 which can provide non-volatile storage of computer code, computer readable instructions, data structures, program modules, and other data for the computer 101. For example and not meant to be limiting, a mass storage device 104 can be a hard disk, a removable magnetic disk, a removable optical disk, magnetic cassettes or other magnetic storage devices, flash memory cards, CD-ROM, digital versatile disks (DVD) or other optical storage, random access memories (RAM), read only memories (ROM), electrically erasable programmable read-only memory (EEPROM), and the like.

Optionally, any number of program modules can be stored on the mass storage device 104, including by way of example, an operating system 105 and prescription module software 106. Each of the operating system 105 and prescription module software 106 (or some combination thereof) can comprise elements of the programming and the prescription module software 106. Prescription and/or treatment data 107 can also be stored on the mass storage device 104. Prescription and/or treatment data 107 can be stored in any of one or more databases known in the art. Examples of such databases comprise, DB2®, Microsoft® Access, Microsoft® SQL Server, Oracle®, mySQL, PostgreSQL, and the like. The databases can be centralized or distributed across multiple systems.

In another aspect, the user can enter commands and information into the computer 101 via an input device (not shown). Examples of such input devices comprise, but are not limited to, a keyboard, pointing device (e.g., a “mouse”), a microphone, a joystick, a scanner, tactile input devices such as gloves, and other body coverings, and the like These and other input devices can be connected to the processing unit 103 via a human machine interface 102 that is coupled to the system bus 113, but can be connected by other interface and bus structures, such as a parallel port, game port, an IEEE 1394 Port (also known as a Firewire port), a serial port, or a universal serial bus (USB).

In yet another aspect, a display device 111 can also be connected to the system bus 113 via an interface, such as a display adapter 109. It is contemplated that the computer 101 can have more than one display adapter 109 and the computer 101 can have more than one display device 111. For example, a display device can be a monitor, an LCD (Liquid Crystal Display), or a projector. In addition to the display device 111, other output peripheral devices can comprise components such as speakers (not shown) and a printer (not shown) which can be connected to the computer 101 via Input/Output Interface 110.

The computer 101 can operate in a networked environment using logical connections to one or more remote computing devices 114a,b,c. By way of example, a remote computing device can be a personal computer, portable computer, a server, a router, a network computer, a peer device or other common network node, and so on. Logical connections between the computer 101 and a remote computing device 114a,b,c can be made via a local area network (LAN) and a general wide area network (WAN). Such network connections can be through a network adapter 108. A network adapter 108 can be implemented in both wired and wireless environments. Such networking environments are conventional and commonplace in offices, enterprise-wide computer networks, intranets, and the Internet 115.

For purposes of illustration, application programs and other executable program components such as the operating system 105 are illustrated herein as discrete blocks, although it is recognized that such programs and components reside at various times in different storage components of the computing device 101, and are executed by the data processor(s) of the computer. An implementation of prescription software 106 can be stored on or transmitted across some form of computer readable media. Computer readable media can be any available media that can be accessed by a computer. By way of example and not meant to be limiting, computer readable media can comprise “computer storage media” and “communications media.” “Computer storage media” comprise volatile and non-volatile, removable and non-removable media implemented in any method or technology for storage of information such as computer readable instructions, data structures, program modules, or other data. Exemplary computer storage media comprises, but is not limited to, RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by a computer.

In various aspects, it is contemplated that the methods and systems described herein can employ Artificial Intelligence techniques such as machine learning and iterative learning. Examples of such techniques include, but are not limited to, expert systems, case based reasoning, Bayesian networks, behavior based AI, neural networks, fuzzy systems, evolutionary computation (e.g. genetic algorithms), swarm intelligence (e.g. ant algorithms), and hybrid intelligent systems (e.g. expert inference rules generated through a neural network or production rules from statistical learning).

The ultraviolet light source 20, illustrated in FIG. 2, can be a conventional ultraviolet light source, according to one aspect. In another aspect, the ultraviolet light source can comprise at least one ultraviolet lamp 22. In another aspect, the at least one ultraviolet lamp can be of predetermined size and wattage. In another aspect, the at least one ultraviolet lamp can comprise a plurality of ultraviolet lamps. As can be appreciated by one of skill in the art, the at least one ultraviolet lamp be sized and positioned in order to treat different disorders. It is contemplated that the ultraviolet light source 20 can comprise at least one light-emitting diode (LED) or other light generating source. In another aspect, the phototherapy computer 101 can be integrally formed with the ultraviolet light source.

In one aspect, the at least one sensor 24 can be configured to send a signal to the phototherapy computer 101 representing at least one operating condition of the ultraviolet light source. In another aspect, the at least one sensor can comprise at least one of a temperature sensor, a light sensor, a current sensor, and a proximity sensor.

In one aspect, the at least one ultraviolet lamp 22 and/or the at least one sensor 24 can comprise an electronic identifier configured to identify the component (i.e., the ultraviolet lamp and/or the at least one sensor) to the phototherapy computer 101. If the proper electronic identifier is not present, according to one aspect, the phototherapy computer can prevent the ultraviolet light source from activating. In another aspect, the electronic identifier can prevent a user or other person from changing a component of the ultraviolet light source in order to circumvent the control system described herein. For example, a user cannot replace the ultraviolet lamp 22 with a higher wattage lamp in order to increase the exposure intensity because the replacement lamp would not have the proper electronic identifier. It is contemplated that the proper electronic identifier can be entered into the phototherapy computer by the manufacturer of the phototherapy device and/or a prescribing physician. It is also contemplated that the proper electronic identifier can be modified in the phototherapy computer by the manufacturer of the phototherapy device and/or the prescribing physician when desired, such as for example and without limitation, when the ultraviolet lamp 22 is to be replaced.

The phototherapy computer 101 can comprise a conventional computer, as described above. In another aspect, a phototherapy control system and data stored on the computer can be accessible through different levels of security requiring different passcodes. In another aspect, the passcode can be any number of characters. In one aspect, a manufacturer of the phototherapy device can be assigned a manufacturer passcode allowing access to all data and functions of the phototherapy device. In another aspect, a physician can be assigned a physician passcode allowing access to, for example and without limitation, the phototherapy control system and/or treatment data, such as how long the ultraviolet light source 20 has been activated. In this aspect, the physician passcode can be required to be input into the phototherapy computer and/or a prescribing computer before the physician can create a prescription for a patient. In still another aspect, a patient can be assigned a patient passcode that must be entered before allowing the patient to activate the ultraviolet light source.

In one aspect, the phototherapy computer 101 can prevent the ultraviolet light source from activating or inactivate the ultraviolet light source if the at least one sensor 24 detects an undesired operating condition. In one example, the ultraviolet light source can be inactivated if the phototherapy device is drawing more current than anticipated. In another aspect, the at least one sensor can transmit data representing the ultraviolet phototherapy exposure of the patient to the phototherapy computer to be logged for later retrieval by the prescribing physician. For example, a temperature sensor can transmit data representing the temperature during a patient's session to the phototherapy computer 101 for logging and later analysis by the prescribing physician. In another example, the ultraviolet light source can be inactive if a proximity sensor indicates that a patient is not positioned in a desired location.

The biometric scanner can be coupled to the phototherapy computer and can be configured for scanning at least one biometric parameter of the patient. In one aspect, the biometric scanner can comprise a scanner configured to scan at least one of: a fingerprint, face, hand and palm geometry, iris or retina, and DNA. In another aspect, the biometric scanner can prevent the ultraviolet light source 20 from activating unless the user attempting to activate the ultraviolet light has a positive scan result. Before using the phototherapy device 10 for the first time, a patient can have at least one biometric parameter scanned in the presence of the physician or other authorized person. In one aspect, the first biometric scan can take place on the biometric scanner of the phototherapy device or a separate biometric scanner. In one aspect, if the first scan takes place on a separate biometric scanner, the results of the scan can be transmitted to the phototherapy device electronically over a network, such as the internet or other conventional means of transmitting electronic data, such as email. In another aspect, the results of the scan can be transmitted to the phototherapy device using a portable storage device, such as a CD, DVD, flash memory card, USB flash drive, floppy disc and the like. Scans subsequent to the first scan can be compared to the first scan, and if the patient is the same, the biometric scanner can indicate a positive result.

In one aspect, a physician can prescribe at least one phototherapy setting for the use of the phototherapy device. In another aspect, the phototherapy setting comprises at least one of: duration of ultraviolet light source activation, frequency of ultraviolet light source activation, timing of ultraviolet light source activation, and intensity of light output. For example, the physician can prescribe that the ultraviolet light source can be activated for a certain amount of time per predetermined time period, such as 10 minutes per day. In another example, the physician can prescribe that the ultraviolet light source can be activated at a predetermined frequency, such as twice daily, every three days, once weekly and the like. In another example, the physician can prescribe that the ultraviolet light source can be activated only at a predetermined time, such as at 3:00 pm on Monday. In still another example, the physician can prescribe that the ultraviolet light source can be activated at a certain intensity level, such that the patient is exposed to the intensity level of ultraviolet light that the physician selects. It is of course contemplated that any number of these and other phototherapy settings can be prescribed alone or in various combinations.

In one aspect, a prescription can be comprised of the at least one phototherapy setting such that the phototherapy device 10 can be used in a remote location from the physician, or otherwise unsupervised setting. In another aspect, the prescription can be selected from a menu of treatment choices, such that the physician is not required to create a new prescription for each patient unless he chooses to do so. The prescription can further comprise a patient and physician identifier, such as the name of the patient and physician, and/or the biometric scan of the patient. In another aspect, the physician can create the prescription directly in the phototherapy computer. In this aspect, the physician can be required to enter the physician passcode and/or be biometrically scanned before accessing the phototherapy computer 101. In another aspect, in order to enter a prescription and/or retrieve logged data from the phototherapy computer, the physician must first enter the physician passcode into the phototherapy computer. In still another aspect, it is contemplated that the prescription can be revised by the physician and the prescribing computer can generate a unique revision code that can be input into the phototherapy computer to indicate the revised prescription. In this aspect, the unique revision code can be transmitted to the phototherapy device remotely over a network, such as the internet or other conventional means of transmitting electronic data, such as email. In another aspect, the unique revision code can be transmitted from the physician to the phototherapy device using a telephone, portable storage device, such as a CD, DVD, flash memory card, USB flash drive, floppy disc and the like.

In another aspect, the physician can write the prescription and print it onto a piece of paper with a prescription number and/or a scanable bar code. In this aspect, the prescription number and/or a scanable bar code can be unique and recognizable only to a phototherapy device that the patient can access.

According to one aspect, the phototherapy device 10 can further comprise the prescribing computer. In this aspect, the physician can enter the prescription for a patient into the prescribing computer. In one aspect, the physician can be required to enter the physician passcode and/or be biometrically scanned before accessing the prescribing computer. The prescription can be transmitted from the prescribing computer to the phototherapy device remotely over a network, such as the internet or other conventional means of transmitting electronic data, such as email. In another aspect, the prescription can be transmitted from the physician to the phototherapy device using a portable storage device, such as a CD, DVD, flash memory card, USB flash drive, floppy disc and the like. In another aspect, the prescribing computer can be linked to at least one phototherapy device, such that a prescription entered into the phototherapy device 10 must be created by the linked prescribing computer.

It is contemplated that the at least one phototherapy setting, patient and physician identifier, and/or any other information being transmitted from the physician to the patient and/or the patient to the physician can be encrypted by conventional means. In order to interpret the encrypted data, in one aspect, there can be a decrypting procedure on the phototherapy computer 101 and/or the prescribing computer. In another aspect, in order to interpret the encrypted data, data from the manufacturer of the phototherapy device and/or the decrypting procedure can be required.

As previously discussed, the patient can have a first biometric scan taken in the presence of the physician or person designated by the physician to witness the scan. The results of the first scan can be transmitted to the phototherapy device 10, if necessary. In order to use the phototherapy device, in one aspect, the patient can enter the patient passcode into the phototherapy computer 101. In another aspect, the patient can scan the appropriate body part into the biometric scanner. If necessary, the patient can provide the prescription to the phototherapy computer by, for example and without limitation, inserting the flash drive containing the prescription into the phototherapy computer. In one aspect, if the patient inputs the proper passcode into the phototherapy computer, if the biometric scanner yields a positive result (i.e., the person being scanned matches the person scanned initially in the presence of the physician), and if the predetermined at least one phototherapy setting is met, the ultraviolet light source can be activated. If the predetermined at least one phototherapy setting is not met, such as, for example and without limitation, if it is not the prescribed time for treatment, the ultraviolet light source 20 will not be activated. Similarly, if the biometric scan results are not positive, or if the wrong passcode is entered into the phototherapy computer 101, the ultraviolet light source will not be activated.

In another aspect, if the patient inputs the proper passcode into the phototherapy computer, if the biometric scanner yields a positive result (i.e., the person being scanned matches the person scanned initially in the presence of the physician), and if the predetermined at least one phototherapy setting is met, the patient can be required to answer certain queries prior to activation of the ultraviolet light source 20. The queries can be assigned by the physician and, depending on the answers entered into the phototherapy computer by the patient, can allow or deny activation of the ultraviolet light source, or can modify the treatment according to a formula preset by the physician. For example, the patient can be required to answer questions such as how the skin of the patient responded to the last exposure, and whether there has been any change in medication, similar to question that might be asked if the treatment was taking place in a clinical setting. If the patient does not provide a predetermined answer to the queries, the ultraviolet light source will not be activated without intervention by the prescribing physician.

When the phototherapy device is being used by the patient, in addition to the queries and the identification process, the phototherapy computer can display instructions or other information for the patient regarding the specifics of the particular treating session, according to one aspect. In another aspect, the patient can be required to type in answers or input a proper key for the treatment to proceed, in order to verify that the patient has read the instructions.

After the ultraviolet light source has been activated, it can remain activated for the amount of time per the phototherapy settings prescribed by the physician before deactivating. Data about the treatment can be logged into the phototherapy computer for later review by the physician. For example, the data about the treatment can comprise use of the device, power interruption, or any other event. After a predetermined number of treatments, or after a predetermined time period, or at other times, in one aspect, the data logged can be transmitted from the phototherapy computer to the prescribing computer and/or to another computer over a network, such as the internet or other conventional means of transmitting electronic data, such as email. In another aspect, the data logged can be can be transmitted from the phototherapy computer to the prescribing computer and/or to another computer using a portable storage device, such as a CD, DVD, flash memory card, USB flash drive, floppy disc and the like. Thus, the physician can have access to all data regarding the use of the device and can manipulate and mine this data as required to improve the treatment received by the patient.

In one aspect, the patient can vary a clock of the phototherapy device 10 by several hours from the time set in the device by the manufacturer. This can allow the patient to update the clock for time zone changes and/or daylight savings time changes. In another aspect, the patient could not, however, reset the clock back 24 hours to attempt to take a missed treatment.

In one aspect, power interruptions while the device is in use can have no effect on the memory of the phototherapy computer 101. For example, momentary interruptions can cause the device to go into a pause mode supported by battery power. If the power is not restored to the phototherapy device in a timely manner, the treatment session can be aborted.

In another aspect, a patient undergoing a treatment session can pause the session for up to a predetermined time period. In another aspect, the physician can set in the prescription a maximum number and/or length of pauses allowed by the patient. If the maximum number and/or length of pauses by the patient is exceeded, the treatment session can be aborted.

In one aspect, if the patient misses a scheduled treatment, or aborts a treatment prior to its completion, the ultraviolet light source can be inactivated until the physician has intervened.

It is contemplated that a single phototherapy device 10 can be used by more than one patient, even if each patient has a different physician because the patient passcode and biometric scan verify the proper prescription is given to each patient. Further, the physician passcode and biometric scan ensure that the treatment data viewable to each physician relates only to the treatment data appropriate to a particulate patient.

In one aspect, because only the physician with an authorized prescribing computer for a given phototherapy device 10 can issue a prescription, the patient cannot double up on exposures by seeing multiple doctors. If a physician stops practicing, or if a patient chooses to change physicians, in another aspect, the manufacture of the phototherapy device can link the phototherapy device to a different physician and/or a different prescribing computer. This can cancel the link between the phototherapy device and the original physician and/or the original prescribing computer.

Although reference has been made herein to an ultraviolet phototherapy control system, it is contemplated that the elements of the system can be used in other applications. For example and without limitation, fluid systems, pill and other medication dispensers, animal feed dispensers, and other manufacturing processes could be controlled with the system described herein.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A phototherapy device configured to control ultraviolet phototherapy exposure to a patient to a desired exposure prescribed by a physician, comprising:
an ultraviolet light source;
a phototherapy computer configured to activate the ultraviolet light source; and
a biometric scanner for scanning at least one biometric parameter of the patient,
wherein the ultraviolet light source, the phototherapy computer and the biometric scanner are electrically coupled, wherein the physician prescribes at least one phototherapy setting, wherein the physician provides at least a proper passcode to the patient, and wherein the at least a proper passcode being input into the phototherapy computer by the patient and a positive result from the biometric scanner are required for the phototherapy computer to activate the ultraviolet light source according to the at least one phototherapy setting.

2. The phototherapy device of claim 1, wherein the phototherapy computer is integral with the ultraviolet light source.

3. The phototherapy device of claim 1, wherein the at least one phototherapy setting comprises at least one of duration of ultraviolet light source activation, frequency of ultraviolet light source activation, timing of ultraviolet light source activation, and intensity of light output.

4. The phototherapy device of claim 3, wherein the at least one phototherapy setting is transmitted to the phototherapy device electronically over a network.

5. The phototherapy device of claim 3, wherein the at least one phototherapy setting is transmitted to the phototherapy device electronically by a portable storage device.

6. The phototherapy device of claim 3, wherein the at least one phototherapy setting is encrypted.

7. The phototherapy device of claim 1, wherein conditions of the activation of the ultraviolet light source are logged by the phototherapy computer and are accessible to the prescribing physician.

8. The phototherapy device of claim 1, wherein the ultraviolet light source comprises an ultraviolet lamp.

9. The phototherapy device of claim 8, further comprising at least one sensor configured to send a signal to the phototherapy computer representing at least one operating condition of the ultraviolet light source

10. The phototherapy device of claim 9, wherein at least one of the ultraviolet lamp and the at least one sensor have an electronic identifier, and wherein the phototherapy computer prevents the ultraviolet light source from activating if the proper electronic identifier is not present.

11. The phototherapy device of claim 9, wherein the phototherapy computer prevents the ultraviolet light source from activating if the at least one sensor signals an undesired operating condition.

12. The phototherapy device of claim 9, wherein the at least one sensor transmits data of the ultraviolet phototherapy exposure of the patient to the phototherapy computer to be logged and wherein the data is accessible to the prescribing physician.

13. The phototherapy device of claim 9, wherein the at least one sensor comprises a temperature sensor.

14. The phototherapy device of claim 9, wherein the at least one sensor comprises a light sensor.

15. The phototherapy device of claim 9, wherein the at least one sensor comprises a current sensor.

16. The phototherapy device of claim 9, wherein the at least one sensor comprises a proximity sensor.

17. A phototherapy system configured to control ultraviolet phototherapy exposure to a patient to a desired exposure prescribed by a physician, comprising:
a phototherapy device comprising:
an ultraviolet light source comprising an ultraviolet lamp;
a phototherapy computer configured to activate the ultraviolet light source;
a biometric scanner for scanning at least one biometric parameter of the patient; and
at least one sensor configured to send a signal to the phototherapy computer representing at least one operating condition of the ultraviolet light source,
wherein the ultraviolet light source, the phototherapy computer and the biometric scanner are electrically coupled, and wherein at least a proper patient passcode input into the phototherapy computer by the patient, a positive result from the biometric scanner, and at least one prescribed phototherapy setting being met are required for the phototherapy computer to activate the ultraviolet light source; and
a prescribing computer,
wherein the physician inputs a physician passcode and the at least one phototherapy setting into the prescribing computer, and wherein the at least one phototherapy setting is transmitted electronically to the phototherapy computer.

18. The phototherapy system of claim 17, wherein the at least one phototherapy setting comprises at least one of duration of ultraviolet light source activation, frequency of ultraviolet light source activation, intensity of light output, and timing of activation.

19. The phototherapy system of claim 17, wherein conditions of the activation of the ultraviolet light source are logged by the phototherapy computer and transmitted electronically to the prescribing computer.

20. The phototherapy system of claim 17, wherein at least one of the at least one ultraviolet lamp and the at least one sensor have an electronic identifier, and wherein the phototherapy computer prevents the ultraviolet light source from activating if the proper electronic identifier is not present.

21. A phototherapy device configured to control ultraviolet phototherapy exposure to a patient to a desired exposure prescribed by a physician, comprising:
an ultraviolet light source comprising at least one ultraviolet lamp;
a biometric scanner for scanning at least one biometric parameter of the patient,
a phototherapy computer electrically coupled to the ultraviolet light source and the biometric scanner; and
means for activating the ultraviolet light source upon the occurrence of at least one parameter established by the prescribing physician.

22. The phototherapy device of claim 21, wherein the at least one parameter comprises at least one of a proper passcode being input into the phototherapy computer by the patient, a positive result from the biometric scanner, and the meeting of predetermined phototherapy settings.

23. The phototherapy device of claim 22, wherein the predetermined phototherapy settings comprise at least one of duration of ultraviolet light source activation, frequency of ultraviolet light source activation, timing of ultraviolet light source activation, and intensity of light output.

24. The phototherapy device of claim 21, further comprising at least one sensor configured to send a signal to the phototherapy computer representing at least one operating condition of the ultraviolet light source.

25. The phototherapy device of claim 24, wherein at least one of the at least one ultraviolet lamp and the at least one sensor have an electronic identifier, and wherein the phototherapy computer prevents the ultraviolet light source from activating if the proper electronic identifier is not present.

26. The phototherapy device of claim 24, wherein the phototherapy computer prevents the ultraviolet light source from activating if the at least one sensor signals an undesired operating condition.

27. The phototherapy device of claim 24, wherein the at least one sensor transmits data of the ultraviolet phototherapy exposure of the patient to the phototherapy computer to be logged and wherein the data is accessible to the prescribing physician.
